(19) United States
(12) Patent Application Publication
(10) Pub. No.: US 2018/0338494 A1
(43) Pub. Date: Nov. 29, 2018

(54) An Antimicrobial Cleansing Composition

(71) Applicant: Conopco, Inc., d/b/a UNILEVER, Englewood Cliffs, NJ (US)

(72) Inventors: Ajit Manohar AGARKHED, Mumbai (IN); Yatin GARG, Sangrur (IN); Swapnil Ravikant HEGISHTE, Mumbai (IN); Nikita TOMAR, Nagpur (IN); Guohui WU, Woodbridge, CT (US)

(73) Assignee: Conopco, Inc., d/b/a UNILEVER, Englewood Cliffs, NJ (US)

(21) Appl. No.: 15/777,940

(22) PCT Filed: Nov. 7, 2016
(86) PCT No.: PCT/EP2016/076842

§ 371 Date: May 22, 2018

(30) Foreign Application Priority Data
Nov. 27, 2015 (EP) EP 15196704.9

(51) Int. Cl. A01N 43/40; A01N 31/08; A01N 31/04; A61Q 19/10; A61Q 17/00; A61K 8/34; A61K 8/49; C11D 3/20; C11D 3/34; C11D 3/48

(52) U.S. Cl. CPC A01N 43/40; A01N 31/08; A01N 31/04; A61Q 19/10; A61Q 17/005; C11D 3/48; A61K 8/34; A61K 8/4933; C11D 3/2037; C11D 3/349; A61K 8/347

(57) ABSTRACT

The present invention relates to an antimicrobial composition comprising 1% to 90% by weight surfactant; a polyvalent metal salt of pyrithione; 0.001 to 3% by weight thymol, and 0.001 to 3% by weight terpineol.

### FIELD OF THE INVENTION

The present invention relates to an antimicrobial composition. It particularly relates to an antimicrobial composition for personal, cleansing.

### BACKGROUND OF THE INVENTION

Human health is impacted by variety of microbes such as protozoans, bacteria, fungi, molds and viruses. For example, invasion by microbial entities including various viruses and bacteria cause a wide variety of diseases and ailments. To reduce the effects of such an invasion, people frequently wash their skin with antimicrobial soaps. Antibacterial soaps typically include soap, which is sodium or potassium salt of fatty acids, in combination with one or more antimicrobial agents like triclosan.

Insoluble particulate metal pyrithiones are acknowledged as antimicrobial agents which are usually incorporated into antimicrobial compositions, such as antidandruff hair shampoos and conditioners.

The constant threat of bacterial contamination and the associated repercussions on health have made antimicrobial solutions a ubiquitous part of commercial and residential cleaning and disinfection processes. The usual cleansing compositions show no detectable reduction in bacterial levels on surfaces amenable to bacterial growth and proliferation in susceptible environments, such as hospitals and in residential kitchen and bath areas. On the other hand, some cleansing compositions produce substantial reduction in bacterial levels but it is generally short-lived. This often results in recontamination due to reuse of such surfaces, requiring frequent reapplication of the disinfectant. Further, relatively high concentrations of the active agent have to be incorporated in such formulations to obtain broad-spectrum disinfection. These high concentrations often have undesirable side effects such as skin and eye irritation, in addition to being potentially hazardous when in contact with food.

WO11/151171 A1 (Unilever) discloses an invention in the field of skin hygiene, especially hand hygiene and/or hand soap compositions. The compositions comprising lower amount of essential oil and a polymer complex or mixture provides improved hygiene efficacy.

WO2004/006876 A1 (Unilever) discloses that tulsi oil or a component compound thereof and a synthetic antimicrobial agent are capable of exhibiting a synergistic antimicrobial activity and are, therefore useful in a composition for treating and/or preventing dandruff. A hair and/or scalp treatment composition comprising tulsi oil and a metal pyrithione are disclosed, wherein the tulsi oil and the metal pyrithione are capable of exhibiting synergistic antimicrobial activity. Tulsi oil comprises high amounts of eugenol.

WO04/035723 A1 (RECKITT BENCKISER) discloses a non-cationic antimicrobial agent containing composition which blooms when added to water. The compositions have good cleaning, disinfecting and bloom properties.

WO9855099 A1 (P&G) discloses a rinse-off antimicrobial cleansing composition comprising 0.1 to 5% of an antimicrobial active, 8 to 18% anionic surfactant, 2 to 12% proton donating agent; 1 to 30% lipophilic skin moisturizing agen, 0.1 to 4% of a stabilizer; and 35 to 88.8% water. At least about 67 percent of the anionic surfactant comprises a mixture of Class A and Class C surfactant. The weight ratio of Class A surfactant to Class C surfactant ranges from about 5:1 to about 1:2. The composition is adjusted to a pH of from about 3.5 to about 4.5.

WO11036048 A1 (Unilever) discloses a method of disinfecting a surface and to antimicrobial composition, particularly for personal cleaning, oral care, or hard surface cleaning. It discloses a method having rapid antimicrobial action. Small amount of eugenol in combination with thymol and terpineol enables this fast kinetics at much lower amount of thymol and terpineol than without eugenol.

WO10046238 A1 (Unilever) discloses an antimicrobial composition for cleansing or personal care. Compositions comprising selected ingredients, namely thymol and terpineol, in selective proportions provide rapid antimicrobial action.

There is need for newer disinfecting compositions that provide sustained broad-spectrum microbial disinfection on surfaces over, prolonged periods without reapplication, even after being contacted by cleaning solutions and after surface reuse.

Furthermore, it is desirable to achieve disinfecting action using lower levels of antimicrobial agents that generally do not pose toxicity-related problems for the user.

Despite a lot of development in the area of cleansing compositions, there is still an unmet need for a personal cleansing composition that provides prolonged antimicrobial efficacy.

There is also a need to provide an efficacious antimicrobial cleansing composition which is also mild for use as a personal wash.

### SUMMARY OF THE INVENTION

According to the first aspect of the present invention, there is provided an antimicrobial composition as claimed in claim 1.

According to another aspect of the invention there is provided a method of inhibiting microbial growth on a surface, said method comprising the steps of applying a composition of the first aspect of the invention to the surface and rinsing the surface with a suitable solvent.

According to yet another aspect is provided the use of a composition according to the first aspect of the invention for improved personal hygiene.

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about”. Unless specified otherwise, numerical ranges expressed in the format “from x to y” are understood to include x and y. When for a specific feature multiple preferred ranges are described in the format “from x to y”, it is understood that all ranges combining the different endpoints are also contemplated.

The present invention provides an antimicrobial composition comprising surfactant in the range of 1% to 90% by weight of the composition; polyvalent metal salt of pyrithione; 0.001 to 3% by weight thymol, and 0.001 to 3% by weight terpineol.

The compositions of the present invention are capable of providing antimicrobial efficacy for preferably at least upto 1 hour and more preferably for at least upto 3 hours, and most preferably for at least upto 5 hours. This time is counted from the time of application of the composition to the concerned substrate, whether animate or inanimate.

Alternatively, compositions of the present invention are capable of providing antimicrobial efficacy which may extend upto 12 hours, more preferably 18 hours and most preferably up to 24 hours.

The present inventors have surprisingly found that compositions comprising ingredients, namely thymol and terpineol and polyvalent metal salts of pyrithione, in selective proportions provide sustained antimicrobial action.

The ingredients of the antimicrobial composition according to the invention are described below. The compositions are preferably meant for non-therapeutic use but may be used for therapeutic purpose. Compositions in accordance with this invention are useful for cleaning animate as well as inanimate surfaces. They are more particularly preferred for cleaning human body including skin, hair and oral cavity. Alternatively, the compositions are useful for cleaning hard surfaces (inanimate).

Surfactant

The composition of the invention comprise 1% to 90% by weight surfactant. This could be a single surfactant but usually it is a combination of various types of surfactants aggregating to the claimed percentage.

In applications meant for personal cleansing, it is preferred that the surfactant is a nonionic surfactant, such as C₈-C₂₂, preferably C₈-C₁₆ fatty alcohol ethoxylates, comprising between 1 and 8 ethylene oxide groups, especially when the product is in the liquid form.

Alternatively, the surfactant is a non-soap anionic surfactant. The term non-soap surfactant is well known in the art and is used to distinguish the anionic surfactants based on their origin/composition.

The non-soap surfactants are preferably selected from primary alkyl sulphates, secondary alkyl sulphates, alkyl benzene sulphonates, or ethoxylated alkyl sulphates. Suitable examples include alkyl ether sulphates, preferably those having 1 to 3 ethylene oxide groups. Especially preferred are sodium lauryl ether sulphates.

Alkyl polyglucosides may also be present in the compositions, preferably those having a carbon chain length between C6 and C16.

Thus, in a highly preferred aspect, the antimicrobial compositions include the surfactant selected from the group of anionic surfactants, alkyl sulphate, fatty acid amide, linear alkyl benzene sulphonate, alkyl amine oxide, CAPB, alkyl glucosides and combinations thereof.

The antimicrobial composition preferably comprises 10 to 90% surfactant by weight of the composition and more preferably from 15 to 85% by weight of the composition. In general, the surfactants may be chosen from the surfactants described in well-known textbooks like “Surface Active Agents” Vol. 1, by Schwartz & Perry, Interscience 1949, Vol. 2 by Schwartz, Perry & Berch, Interscience 1958, and/or the current edition of “McCutcheon's Emulsifiers and Detergents” published by Manufacturing Confectioners Company or in “Tenside-Taschenbuch”, H. Stache, 2nd Edn., Carl Hauser Verlag, 1981. Any type of surfactant, i.e. anionic, cationic, nonionic, zwitterionic or amphoteric can be used.

Anionic surfactants are particularly preferred. It is further preferred that the surfactant is a soap. Soap is a surfactant suitable for personal human hygiene. When the anionic surfactant is a soap, it is preferred that the soap is C₈-C₂₄ soap, more preferably C₂ soap and most preferably C₁₂-C₁₈ a soap. The soap may or may not h have unsaturation. The cations in the soap molecules can be alkali metal, alkaline earth metal or ammonium ions. Preferably, the cation is sodium, potassium or ammonium. More preferably the cation is sodium or potassium.

The soap is usually obtained by saponifying a fat and/or a fatty acid. The fats or oils generally used in soap manufacture may be such as tallow, tallow stearines, palm oil, palm stearines, soya bean oil, fish oil, castor oil, rice bran oil, sunflower oil, coconut oil, babassu oil, palm kernel oil and others. In the above process, the fatty acids are derived from oils/fats selected from coconut, rice bran, groundnut, tallow, palm, palm kernel, cottonseed, soya bean and castor. The fatty acid soaps can also be synthetically prepared (e.g. by the oxidation of petroleum or by the hydrogenation of carbon monoxide by the Fischer-Tropsch process). Resin acids, such as those present in tall oil, may be used. Naphthenic acids are also suitable.

Tallow fatty acids can be derived from various animal sources and generally comprise about 1 to 8% myristic acid, about 21 to 32% palmitic acid, about 14 to 31% stearic acid, about 0 to 4% palmitoleic acid, about 36 to 50% oleic acid and about 0 to 5% linoleic acid. A typical distribution is 2.5% myristic acid, 29% palmitic acid, 23% stearic acid, 2% palmitoleic acid, 41.5% oleic acid, and 3% linoleic acid by weight of soap. Other similar mixtures, such as those from palm oil and those derived from various animal tallow and lard are also included.

Coconut oil refers to fatty acid mixtures having an approximate carbon chain length distribution of 8% C₈, 7% C₁₀, 48% C₁₂, 17% C₁₄, 8% C₁₆, 2% C₁₈, 7% oleic and 2% linoleic acids (the first six fatty acids listed being saturated) by weight of soap. Other sources having similar carbon chain length distribution, such as palm kernel oil and babassu kernel oil, are included within the term coconut oil.

A typical fatty acid blend consisted of 5 to 30% coconut fatty acids and 70 to 95% fatty acids by weight of soap. Fatty acids derived from other suitable oils/fats such as groundnut, soybean, tallow, palm and palm kernel may also be used in other desired proportions.

It is preferred that compositions in accordance with the invention comprise 25 to 90% by weight soap, more preferably from 50 to 85% and most more preferably 55 to 75% by weight of the composition.

The antimicrobial composition may be in form of a solid, a liquid, a gel or a paste. A person skilled in the art can prepare compositions in various formats by choosing one or more additional ingredients and/or surfactant. Reference may be made to standard textbook and encyclopedias. The antimicrobial compositions of the present invention are useful for cleansing and care, in particular for skin cleansing.

It is preferred that the antimicrobial cleansing compositions of the invention are i flowable.

When the composition is flowable it preferably comprises 0.05 to 3% by weight thymol, 0.05 to 3% by weight terpineol, 0.001% to 3% by weight zinc pyrithione, and 1 to 45% by weight surfactant.

Preferred flowable compositions of the present invention may further comprise 10% to 99.9% by weight water.

Plowable antimicrobial compositions are useful for skin cleansing, in particular for hand wash, body wash or as a face wash.

Alternatively, the antimicrobial cleansing compositions are solid compositions. Such compositions are non-flowable. When the composition is solid, it preferably comprises 0.05 to 3% by weight thymol, 0.05 to 3% by weight terpineol, 0.001% to 3% by weight zinc pyrithione, and 20 to 90% by weight surfactant.

Preferably, the solid compositions of the present invention comprise 5% to 30% by weight water.

The solid antimicrobial composition is preferably in a shaped solid form, more preferably a bar or cake of soap. The solid antimicrobial composition is particularly useful for skin cleansing in particular for bathing, hand wash, or face wash.

Thymol

The antimicrobial compositions of the invention, comprise 0.001 to 3%, more preferably 0.01 to 1%, and most preferably 0.05 to 0.6% by weight thymol. Most of the useful antimicrobial compositions of the present invention have thymol higher than 0.05 but lesser than 0.8% by weight. These ranges are preferred because below the preferred lower concentration of thymol, the desired faster acting antimicrobial kinetics may not be met under all circumstances. At concentrations higher than the higher range, while the kinetics of action would not be compromised in combination with terpineol the sensorial aspects like smell and skin feel could be compromised. Thymol may be added to the antimicrobial composition in purified form.

Alternatively, a suitable amount of thyme oil or thyme extract comprising the desired amount of thymol may be included, Thyme oil or thyme extract is obtained from the thyme plant, belonging be genus Thymus and includes but is not limited to the following species: Thymus vulgaris, Thymus zygis, Thymus satureoides, Thymus mastichina, Thymus broussonetti, Thymus maroccanus, Thymus pallidus, Thymus algeriensis, Thymus serpylium, Thymus pulegoide, and Thymus citriodorus.

The structures of thymol and its isomer carvacrol are given below:

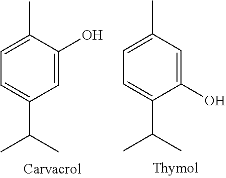

Terpineol

The antimicrobial compositions of the invention comprise 0.001 to 3%, more preferably 0.05 to 1%, and most preferably 0.2 to 0.6% by weight terpineol. Most of the useful fast acting antimicrobial compositions of the present invention have terpineol higher than 0.05 but lesser than 1% by weight terpineol. These preferred concentration ranges of terpineol are important for the reasons indicated in the context of thymol. The terpineol is preferably selected from alpha-terpineol, beta-terpineol, gamma-terpineol or mixtures thereof. It is particularly preferred that the terpineol is alpha-terpineol. Terpineol may be added to the antimicrobial composition in purified form.

Alternatively, an amount of pine oil comprising desired amount of terpineol in it may be added to the antimicrobial composition.

The structure of terpineol compound is given below:

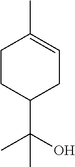

It is preferred that the sum of percentages of Thymol and Terpineol (by weight) is from 0.01 to 2%, more preferably from 0.1 to 2% and most preferably from 0.1 to 1% by weight of the composition.

It is particularly preferred that antimicrobial compositions of the invention comprise thymol and terpineol at w/w ratio of 1:1.

Polyvalent Metal Salt of Pyrithione

The insoluble metal pyrithione may be represented by the following general formula:

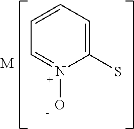

in which M is a polyvalent metal ion and n corresponds to the valency of M.

Preferred examples of M include magnesium, barium, strontium, zinc, cadmium, tin and zirconium. Especially preferred is zinc.

The polyvalent metal salt of pyrithione may have any particle form suitable for use in a composition for topical application to the skin. For example, the metal pyrithione may be in the form of amorphous or crystalline particles having a range of different particle sizes.

The polyvalent metal salt of pyrithione may, for example, be in the form of particles having size distribution in which at least about 90% of the particles have a size of up to 100 μm, more preferably up to 50 μm, even more preferably up to 10 μm, most preferably 5 μm or less, for example the size distribution may be such that at least about 90% of the particles have a size of 1 μm or less In particular for hair compositions, smaller sizes are optimal for antimicrobial effect.

Various methods for producing fine particles of polyvalent metal salt of pyrithione are described, for example, in EP0173259 B1 (Kao Corp, 1991). Suitable methods for determining particle size are described in that publication.

The polyvalent metal salt of pyrithione may be made up of one particulate form or two or more different particulate forms.

Other suitable particulate forms for the polyvalent metal salt of pyrithione include platelets and needle-shaped particles. Platelets of zinc pyrithione are described in EP 0034385 B1 (P&G, 1984). The needle-shaped particles are preferably of the type described in WO99/66886 A1 (Unilever, 1999). For needle-shaped particles, preferably at least 50% of the particles are needle-shaped particles having a length of between 1 μm and 50 μm.

A preferred amount of polyvalent metal salt of pyrithione for the composition of the present invention is from about 0.001% to about 5% by weight of the total composition, more preferably from about 0.05% to about 3% by weight, most preferably between 0.1% and 1% by weight. It is preferred that the polyvalent metal salt of pyrithione is Zinc pyrithione.

Additional Ingredients

Preferred compositions may include other known ingredients such as perfumes, pigments, preservatives, emollients, sunscreens, emulsifiers, gelling agents and thickening agents. Choice of these ingredients will largely depend on the format of the composition.

The inventors have determined that while a combination of thymol and terpineol alone do provide the fast antimicrobial kinetic action, a combination of thymol and terpineol along with the polyvalent metal salt of pyrithione provides synergistic sustained antimicrobial action which is especially important in a wash-off product (as opposed to a leave-on product) where the contact time of the antimicrobial actives with the surface is low. Fortuitously, such wash off processes include a surfactant for the cleaning action. While the surfactant alone does not provide sustained antimicrobial effect at their typical concentrations present in wash-off processes, it provides further improvement in the extent of reduction in the microbial count on the surface for a period of time when the surfaces are washed/treated with a composition in accordance with the invention.

Daily use of antibacterial soap for personal cleansing controls the growth of pathogenic bacteria on our skin. This in turn prevents at least some skin diseases. The surfactants present in such compositions remove bacteria and other microbes lodged on the skin, while deposition of an active ingredients that can control the growth and colonisation of organisms which are left behind.

In another aspect of the present invention, the composition of the present invention is suitable for use in wipes (in impregnated form) for personal hygiene or surface cleaning such as for household care.

According to another aspect of the present invention there is provided a method of disinfecting a surface comprising the steps of applying a composition of the first aspect of the invention on to the surface; and rinsing the surface with a suitable solvent.

The solvent for rinsing the surface is preferably water but could also be a mixture of water and alcohol. The word rinsing herein includes the act of wiping the surface with a suitable wipe. Thus, the surface e.g. hand, face, body, oral cavity or any hard surface e.g. a utensil is first contacted with the composition of the invention. It is then rinsed preferably with sufficient amounts of water after a pre-determined period of time to remove any visible or sensory reside of the composition. Alternatively, an alcohol wipe or a water/alcohol impregnated wipe may be used to wipe the surface to be visibly free of the anti-microbial composition. The step of rinsing the substrate is preferably carried out less than 5 minutes, preferably less than 2 minutes, further more preferably less than a minute and in many cases less than 15 seconds after the step of applying the composition on the substrate.

The surface is an animate surface such as human body or any part thereof. Alternatively, the surface is an inanimate surface such as table-tops and tiles. When the surface is animate, it is preferred that the method is-non-therapeutic. Such methods include cosmetic methods.

Alternatively, the method is therapeutic. Treatment by therapy is defined as any treatment which is designed to cure, alleviate, remove or lessen the symptoms of, or prevent or reduce the possibility of contracting any disorder or malfunction of human or animal body. According to one aspect, the invention provides for non-therapeutic benefits.

One aspect of the present invention provides use of the composition of present invention for improved personal hygiene.

The invention also provides for therapeutic benefits.

The composition of the present invention is suitable for use as an anti-dandruff treatment.

### EXAMPLES

The invention will be demonstrated with examples. The examples are for the purpose of illustration only and they do not limit the scope of claims in any manner.

The examples were conducted by adding various actives on the following base composition of soap bars:

TABLE 1
Base Composition of the soap bars

| Ingredients | % by weight |
|---|---|
| Soap | 72.0 |
| Glycerine | 4.0 |
| Primary Alkyl Sulphate (PAS) | 2.0 |
| Sodium Chloride | 0.7 |
| Talc | 8.0 |
| Water and other minor ingredients made up to 100% | — |
| Total | 100.0 |

### Example 1: Antimicrobial Efficacy of the Composition

Preparation of Bacteria (E. coli Culture)

Escherichia coli ATCC 10536 was obtained as a lyophilized culture from American Type Culture Collection. The test culture was grown for 24 h on Tryptic Soy Agar (TSA) streak plate at 37.0° C. Then E. coli suspension was prepared at 1˜5×10⁷ CFU/ml with Tryptone Sodium Chloride right before the efficacy tests.

Sustained Antimicrobial Efficacy Test

To determine efficacy of an antimicrobial soap bar, in-vitro performance tests were performed on artificial skin samples (VITRO-SKIN™, IMS Corp., a synthetic substrate designed to mimic the surface chemistry of human skin). To prepare the substrate, pieces of VITRO-SKIN™ were hydrated overnight in a hydration chamber with a reservoir of 85% water and 15% glycerin. After approximately 24 hours, the VITRO-SKIN pieces were taken out of the chamber and allowed to rest at ambient temperature and humidity for approximately one hour, Five cm diameter sections were mounted between the opposing pieces of an XRF cup.

To mimic washing the skin, the soap bar was cut into a cylinder of 1 cm diameter which was wetted with deionised water, the bar soap composition gently rubbed across the entire VITRO-SKIN™ surface inside the XRF cup for 15 seconds. Then, lather was generated by continuously rubbing the VITRO-SKIN™, with a Teflon rod for 45 seconds (e.g. absent the bar soap composition). The wash liquor was removed and the VITRO-SKIN™ was rinsed by adding 4 ml of deionized water to the XRF cup, and the substrate was rubbed with a clean Teflon rod for 30 seconds. The rinse step was repeated one more time. After removing the rinse liquor, the VITRO-SKIN™ was allowed to dry for 5 to 10 minutes in a still room air under low light conditions. Each VITRO-SKIN™ was inoculated evenly with 10⁶ to 10⁷ CFUs E. coli by using 200 μl of culture obtained from an overnight growth as described above. The bacteria were allowed to dry on the VITRO-SKIN for 20 minutes, then the VITRO-SKIN was placed inside an incubator for 0 to 3 hours. After incubation, 10 ml ice cold DIE broth was added into each XRF cup, which was covered with Teflon caps tightly and was vigorously shaken for 1 minute to dislodge the bacteria. Serial dilutions of the fluids were made and were plated for colony count with Tryptic Soy Broth for 24 hours at 37° C. Thereafter, the viable bacteria were counted and reported in the form of log₁₀ CFU. The smaller log₁₀ (CFU/ml) value correspond to more potent and sustained antimicrobial efficacy of a given sample.

TABLE 2
Examples of antimicrobial efficacy

| | Log (Viable E. coli) | |
|---|---|---|
| Details of the composition(s) | 0 time | 3 hour |
| 100% by weight Base Composition only (No zinc | 7.0 | 7.0 |
| pyrithone, No thymol, No terpineol) | | |
| 99.65% by weight Base Composition + 0.35% by | 7.0 | 7.0 |
| weight TT which is (0.175% by weight thymol + | | |
| 0.175% by weight terpineol) | | |
| 99.65% by weight Base Composition + 0.35% by | 7.0 | 6.8 |
| weight ZPTO (zinc pyrithione) | | |
| 99.30% by weight Base Composition + 0.35% by | 7.0 | 6.0 |
| weight ZPTO + 0.35% by weight TT which is | | |
| (0.175% by weight thymol + 0.175% by weight | | |
| terpineol) | | |

Note:
in the above table(s), the term TT is used to collectively represent Thymol and Terpineol

The data in Table 2 show that the composition of the present invention provides longer lasting antimicrobial benefits for up to 3 hours. The data further shows that the surface is disinfected when it is treated with a composition in accordance with the invention (row-4).

1. An antimicrobial cleansing composition comprising:
(a) 1% to 90% by weight surfactant;
(b) polyvalent metal salt of pyrithione;
(c) 0.001% to 3% by weight thymol, and,
(d) 0.001% to 3% by weight terpineol based on total weight of the composition.

2. An antimicrobial composition as claimed in claim 1, wherein the polyvalent metal salt of pyrithione is 0.001% to 6% by weight of the composition.

3. An antimicrobial composition as claimed in claim 1, wherein the polyvalent metal salt of pyrithione is zinc pyrithione.

4. An antimicrobial composition as claimed in claim 1 wherein the surfactant comprises soap.

5. An antimicrobial composition as claimed in claim 1 wherein the terpineol is selected from the group of alpha-terpineol, beta-terpineol, gammaterpineol and mixtures thereof.

6. An antimicrobial composition as claimed in claim 5, wherein said terpineol is alpha-terpineol.

7. An antimicrobial composition as claimed in claim 1 wherein said composition comprises thymol and terpineol at w/w ratio of 1:1.

8. A method of disinfecting a surface, comprising the steps of:
(a) applying a composition as claimed in claim 1 on to the surface;
and,
(b) rinsing the surface with a suitable solvent.

9. A method as claimed in claim 8 wherein said surface is animate or inanimate.

10. A method as claimed in claim 9 wherein when said surface is animate, said method is non-therapeutic.

11. Use of a composition according to claim 1, for improved personal hygiene.
